(19) United States
(12) Patent Application Publication
(10) Pub. No.: US 2002/0045254 A1
(43) Pub. Date: Apr. 18, 2002

(54) Established cells

(76) Inventors: Kenichi Hosoya, Sendai-shi (JP); Tetsuya Terasaki, Sendai-shi (JP); Masatsugu Ueda, Kawagoe-shi (JP); Masuo Obinata, Sendai-shi (JP)

Correspondence Address: PAULA CAMPBELL EVANS, PALMER & DODGE LLP, One Beacon Street, Boston, MA, 02108, US

(21) Appl. No.: 09/821,654

(22) Filed: Mar. 29, 2001

(30) Foreign Application Priority Data
Oct. 1, 1999 (JP) PCT/JP99/05423
Oct. 2, 1998 (JP) 296138-1998
Oct. 2, 1998 (JP) 296139-1998

(51) Int. Cl. C12N 5/06

(52) U.S. Cl. 435/325; 800/21

(57) ABSTRACT

Established cells derived from retinal capillary endothelial cells, choroid plexus epithelial cells, or brain capillary endothelial cells or a transgenic animal carrying a large T-antigen gene of an SV40 temperature sensitive mutant tsA58. The cell line derived from retinal capillary endothelial cells expresses a temperature sensitive SV40 large T-antigen, GLUT-1 transporter, and p-glycoprotein. The cell line derived from choroid plexus epithelial cells expresses a temperature sensitive SV40 large T-antigen gene and shows localization of Na⁺—K⁺ ATPase and GLUT-1 transporter in the cell membrane. When cultured in a monolayer, it shows the localization of Na⁺—K⁺ ATPase in the apical side. The cell line derived from brain capillary endothelial cells expresses a temperature sensitive SV40 large T-antigen, GLUT-1 transporter, p-glycoprotein, alkaline phosphatase, and γ-glutamyltransferase.

A method of establishing immortalized cells by subculturing cells obtained from retinal capillary endothelial cells, choroid plexus epithelial cells, or brain capillary endothelial cells of the above-described transgenic animal. These cells are useful in screening drugs regarding safety and efficacy thereof, and developing method for diagnosing and treating diseases relating to nutrition metabolism in retinal tissues and brain on cellular level studies.

### TECHNICAL FIELD

The present invention relates to immortalized cells established from a transgenic animal into which a large T-antigen gene of SV40 temperature sensitive mutant tsA58 has been introduced.

More specifically, the present invention relates to established cells derived from retinal capillary endothelial cells of the transgenic animal.

The established cells derived from retinal capillary endothelial cells of the present invention form a monolayer of the retinal capillary endothelial cells which have inside-and outside polarity when culturing in a culture dish. Therefore, the established cells are useful for predicting permeation of drugs to the retina by the assessment of chemical uptake into the retinal capillary endothelial cells, studying supply and metabolism of various factors and nutritions in the retinal parenchyma, studying the transport mechanism of permeation of selective materials which are present in retinal capillary endothelial cells, studying toxicity of chemicals on retinal capillary endothelial cells, and so on. In addition, a blood retinal barrier can be reconstructed in the test tube (in vitro) by coculture with Mueller cells which are a kind of glia cells. The cell lines of the present invention are therefore useful in screening drugs regarding the safety and efficacy thereof, and developing methods for diagnosing and treating diseases relating to intraocular homeostatic maintenance and functional disorders of retinal tissues on cellular level studies.

The present invention also relates to established cells derived from choroid plexus epithelial cells of the transgenic animal.

The established cells derived from choroid plexus epithelial cells of the present invention are useful for studying nutrition metabolism in the brain, studying permeation of drugs into the brain, and investigating the protection mechanism of metabolism and permeation of substances into the cerebrospinal system. These cells are therefore useful in screening drugs regarding the safety and efficacy thereof, and developing methods for diagnosing and treating diseases relating to nutrition metabolism disorders and homeostatic functional disorders of the brain on cellular level studies.

Furthermore, the present invention relates to established cells derived from brain capillary endothelial cells of the transgenic animal.

The established cells derived from brain capillary endothelial cells of the present invention are useful for studying the blood-brain barrier which restricts moves of substances from blood to brain tissues. Specifically, these cells are useful for the study of nutrition metabolism in the brain, the study of permeation of chemicals into the brain, and the study of the protection mechanism of metabolism and permeation of substances into the cerebrospinal system. These cells are therefore useful in screening drugs regarding the safety and efficacy thereof, and developing method for diagnosing and treating diseases relating to nutrition metabolism disorders and homeostatic functional disorders of the brain on cellular level studies.

### BACKGROUND ART

Conventionally, tests for the assessment of safety and efficacy of drugs have been conducted using animals. However, to avoid use of a large number of animals from the viewpoint of animal right, technologies for in-vitro assessment of safety and efficacy of drugs using cultured cells are used on a practical level. For example, a technique of first testing using primary culture cells collected from living tissues or established immortalized cells which can infinitely proliferate, and then testing using animals is employed. The primary culture cells can initially proliferate very well, but the proliferation gradually declines as the subculture advances, and finally cells die out. This phenomenon is called cellular senescence. Furthermore, in addition to the fear that the characteristics of primary culture cells may differ each time they are collected from living tissues, the primary culture cells are said to change the characteristics as the subculture advances. Particularly, when the multiplication rate is very slow or when the cells are derived from a small organ, it is very difficult to obtain a sufficient amount of the primary culture cells for test. On the other hand, established culture cell which have acquired the capability of infinitely proliferating during subcultures of the primary culture cells can maintain stable characteristics. However, most of these cells no longer have part or all of the forms and functions possessed by the cells when they were in a living body. Therefore, it is difficult for such established cells to precisely reflect the original characteristics which the cell lines exhibited in the tissues from which they have been derived. In view of this situation, establishment of immortalized cells which can continuously maintain an active proliferation capability possessed by the primary culture cells without losing the characteristics inherently possessed by the cells during subculture, has be entried by transforming the cells by introducing oncogenes such as ras and c-myc, E1A gene of adenovirus, large T-antigen gene of SV40 virus, or HPV16 gene of human papillomavirus. Such immortalized cells which are derived, from some organs lose several functions at the time of introducing oncogenes or large T-antigen genes after preparation of a primary culture cell. Thus, acquisition of immortalized cells in the stringent meaning of holding an original function has been difficult. Preparing a primary culture cell and acquiring a cell line has been very difficult, particularly when the multiplication rate is very slow or when the cells are-derived from a small organization.

To overcome these problems, a method of establishing immortalized cells by applying a recently developed transgenic technology to individual animals has been proposed. Instead of introducing oncogenes or large T-antigen genes into individual cells, according to this method, transgenic animals into which these genes have been introduced in chromosomes in a stable manner are prepared. Then, a primary culture cell is prepared from an organ of these animals which possesses the oncogenes or large T-antigen genes in the cells at the time of development of the individuals. The primary culture cells is subcultured to establish immortalized cells. In particular, immortalized cells are easily available from organs of transgenic mice into which a large T-antigen gene of a temperature sensitive mutant tsA58 of SV40 has been introduced. The immortalized cells are very useful because growth of the resulting cells and expression of the differentiation character can be managed by changing the temperature (Noble M. et al. (1995) Transgenic Research 4, 215-225; ObinataM. (1997) Genes to Cells 2,235-244. Rats having a body weight about ten times that of mice are advantageous for preparing cells used for the establishment of a cell line from various organs, particularly for preparing a cell line originating from small organs such as retinal capillary endothelial cells or intracerebral cells (e.g. choroid plexus epithelial cells, capillary vessel endothelial cells, etc.), because primary culture cells or many other cells can be easily obtained by separating organs or tissues from the rats. Therefore, transgenic rats into which a large T-antigen gene of a temperature sensitive mutant tsA58 of SV40 has been introduced, which are useful for establishing immortalized cells due to easy availability from various organs and the capability of controlling the growth of the resulting cells and expression of the differentiation character by changing temperatures, had already been produced.

On the other hand, a method of using a primary culture cell of retinal capillary endothelium in place of animal tests is being developed in view of animal right. In this instance, because it is difficult to obtain a sufficient amount of primary culture cells which can be for test from small animals, the eyeballs of large animals such as cattle must be used. However, the number of cells obtained by isolating retinal capillary endothelial cells from twenty eyeballs of cattle and subculturing the cells for two generations is at most 9×10⁶ or so (Wong H. C. et al. (1987) Invest. Ophthalmol, Visual, Sci., 28, 1767-1775). Thus, a great number of eyeballs of cattle is required for screening drugs. Therefore, an effective retinal capillary endothelial cell stock which can be used in place of the cells from the eyeballs of cattle has been desired.

For the same reason, in research investigating the effect and mechanism of nerve drugs on the blood-cerebrospinal fluid barrier mechanism, a method of using a primary culture cell of choroid plexus epithelial cells in place of animal tests is being developed in view of animal right. In this instance, because it is difficult to constantly obtain a sufficient amount of culture cells for the test from small animals, effective cell lines usable in place of such culture cells have been strongly desired.

Furthermore, in toxicology research investigating the effect and mechanism of drug transfer into the brain on the blood brain barrier mechanism, a method of using a primary culture cell of cerebrovascular endothelial cells in place of animal tests is being developed in view of animal right. In this instance, because it is difficult to constantly obtain a sufficient of culture cells for the test, valuable cell lines usable in place of such culture cell have been strongly desired.

### DISCLOSURE OF THE INVENTION

In view of this situation, the present inventors have conducted extensive studies and, as a result, have established immortalized cells from transgenic rats into which immortalizing genes have been introduced by separating retinal capillary vessels from the retinal tissue of the rats and isolating retinal capillary endothelial cells from the resulting capillary vessels.

An object of the present invention is therefore to obtain established cells derived from retinal capillary endothelial cells and capable of expressing a temperature sensitive SV40 large T-antigen, GLUT-1 transport carrier, and p-glycoprotein.

Another object of the present invention is to provide a method of establishing immortalized cells using a large T-antigen gene of SV40 temperature sensitive mutant tsA58.

In addition, the present inventors have established immortalized cells from transgenic rats into which immortalizing genes have been introduced by separating an epithelial cell line from the choroid plexus of brain.

Still another object of the present invention is therefore to provide established cells derived from choroid plexus epithelial cells, capable of expressing a temperature sensitive SV40 large T-antigen gene, showing localization of Na⁺—K⁺ ATPase and GLUT-1 transport carriers in the cell membrane, and when cultured in a monolayer, showing the localization of Na⁺—K⁺ ATPase in the apical side.

A further object of the present invention is to provide a method of establishing such immortalized cells using a large T-antigen gene of the SV40 temperature sensitive mutant tsA58.

Still further, the present inventors have established immortalized cells from transgenic rats into which immortalizing genes have been introduced by separating brain capillary vessels from the brain of the rats and isolating brain capillary endothelial cells from the resulting capillary vessels.

A further object of the present invention is therefore to obtain established cells derived from brain capillary endothelial cells and capable of expressing a temperature sensitive SV40 large T-antigen, GLUT-1 transport carrier, p-glycoprotein, alkaline phosphatase, and γ-glutamyltransferase.

A still further object of the present invention is to provide a method of establishing immortalized cells using a large T-antigen gene of SV40 temperature sensitive mutant tsA58.

The present invention has been completed to achieve the above objects and relates to immortalized cells established from a transgenic animal into which a large T-antigen gene of SV40 temperature sensitive mutant tsA58 has been introduced. More specifically, the present invention relates to the established cells derived from retinal capillary endothelial cells of such transgenic animals. In particular, the present invention relates to established cells which express a temperature sensitive SV40 large T-antigen, GLUT-1 transport carrier, and p-glycoprotein. Cell deposited in National Institute of Bioscience and Human-Technology, Agency of Industrial Science and Technology, the Ministry of International Trade and Industries, under the deposition number FERMBP-6507 can be given as such established cells.

Furthermore, the present invention relates to a method of establishing immortalized cells comprising homogenizing the retinal tissue of such a transgenic animal, separating capillary vessels, treating the resulting retinal capillary vessels with protease, and subculturing the resulting cells. The rat can be given as an example of such a transgenic animal.

Furthermore, the present invention relates to the established cell obtained using such a method of establishment.

Such established cells of the present invention form a monolayer of the retinal capillary endothelial cells which have inside-and-outside polarity when culturing in culture dish. Therefore, the established cells are useful for predicting permeation of drugs into the retina by the assessment of drug uptake into the retinal capillary endothelial cells, studying supply and metabolism of various factors and nutritions in the retinal parenchyma, studying the transport mechanism of permeation of selective materials which are present in retinal capillary endothelial cells, studying toxicity of drugs on retinal capillary endothelial cells, and so on. In addition, a blood retinal barrier can be reconstructed in a test tube (in vitro) by coculturing with Mueller cells which are a kind of gliacells. The celllines of the present invention are therefore useful in screening drugs regarding safety and efficacy thereof, and developing a method for diagnosing and treating diseases relating to intraocular homeostatic maintenance and functional disorders of retinal tissues through cellular level studies.

The present invention also relates to established cells derived from choroid plexus epithelial cells of such a transgenic animal. Specifically, the present invention relates to established cells expressing a temperature sensitive SV40 large T-antigen gene, showing localization of Na⁺—K⁺ ATPase and GLUT-1 transport carriers in the cell membrane, and when cultured in a monolayer, showing the localization of Na⁺—K⁺ ATPase in the apical side. The cells deposited in National Institute of Bioscience and Human-Technology, Agency of Industrial Science and Technology, the Ministry of International Trade and Industries, under the deposition number FERMBP-6508 can be given as such established cells.

The present invention also relates to a method of establishing immortalized cells comprising treating the choroidal plexus tissues of such a transgenic animal with protease, selecting the cells exhibiting an epithelial cell-like/paving stone-like form from the resulting cells, and subculturing such cells. The rat can be given as an example of such a transgenic animal.

Furthermore, the present invention relates to the established cells obtained using such a method of establishment.

Due to the capability of forming tight junction among cells when cultured in a mono-layer on a porous flat membrane and the capability of reconstructing the blood-cerebrospinal fluid barrier with a inside-and-outside polarity in vitro, the established cells are useful for studying nutrition metabolism of the brain, studying permeation of drugs into the brain, and investigating the protection mechanism of metabolism and permeation of substances into the cerebrospinal system. These cells are therefore useful in screening drugs regarding the safety and efficacy thereof, and developing a method for diagnosing and treating diseases relating to nutrition metabolism disorders and homeostatic functional disorders of the brain on cellular level studies.

Furthermore, the present invention relates to established cells derived from brain capillary endothelial cells of such a transgenic animal. Specifically, the present invention relates to established cells which express a temperature sensitive SV40 large T-antigen, maintain an alkaline phosphatase activity and γ-glutamyltransferase activity, and express a scavenger receptor,GLUT-1 transporter and p-glycoprotein. The cell line deposited in National Institute of Bioscience and Human-Technology, Agency of Industrial Science and Technology, the Ministry of International Trade and Industries, under the deposition number FERMBP-6873 can be given as such an established cell.

The present invention also relates to a method of establishing immortalized cells comprising separating brain capillary vessels from the brain tissues of such a transgenic animal, treating the brain capillary vessels with protease, selecting the cells exhibiting a spindle fiber-like form specific to endothelial cells from the resulting cells, and subculturing such cells. The rat can be given as an example of such a transgenic animal.

Furthermore, the present invention relates to the established cells obtained using such a method of establishment.

Due to the capability of mutually bonding and reconstructing the blood-brain barrier with a inside-and-outside polarity in vitro when cultured in a mono-layer on a porous flat membrane, the established cells are useful for studying the blood-brain barrier which restricts movement of substances to the brain tissues from blood, specifically, studying the nutrition metabolism in the brain and permeation of drugs into the brain, and investigating the protection mechanism in the blood-brain barrier. These cells are therefore useful in screening drugs regarding the safety and efficacy thereof, and developing a method for diagnosing and treating diseases relating to nutrition metabolism disorders and homeostatic functional disorders of the brain through cellular level studies.

### BRIEF DESCRIPTION OF THE DRAWINGS

FIG. 1 shows a substrate concentration dependency of a 3-OMG uptake speed of the established cell (TR-iBBB2) obtained in Example 5 of the present invention.

FIG. 2 shows confocal laser scanning microscopy of Na⁺—K⁺ ATPase of the established cell (TR-CSFB3) obtained in Example 10 of the present invention.

The upper photograph is a microscopic photograph of a plan view (XY section)of the cell wherein Na⁺—K⁺ ATPase and GLUT-1 are seen to be expressed. The lower photograph is a microscopic photograph of a cross section view (XZ section) of the cell wherein Na⁺—K⁺ ATPase are seen localized in apical side.

FIG. 3 shows the proline active transport capability of the established cell (TR-CSFB3) obtained in Example 11 of the present invention.

FIG. 4 shows interference of the proline active transport capability of the established cell (TR-CSFB3) obtained in Example 12 of the present invention by choline and ouabain.

### BEST MODE FOR CARRYING OUT THE INVENTION

The transgenic rat used in the present invention into which a large T-antigen gene of SV40 temperature sensitive mutant tsA58 has been introduced can be obtained as follows. Specifically, a whole genome DNA of tsA58ori(−)−2 which is produced from a large T-antigen gene of a temperature sensitive mutant tsA58 of SV40, for example, with deletion of the SV40 ori (replication origin), is linearized using a restriction endonuclease BamHI, and introduced into pBR322 to obtain a plasmid pSVtsA58ori(−)−2 (Ohno T. et al., Cytotechnology 7, 165-172 (1991)) pBR322. The plasmid is amplified in Escherichia coli in a large amount according to a conventional method.

The plasmid thus obtained is cut with a restriction endonuclease BamHI to eliminate a vector region. Because the DNA (5,240 bp) having a large T-antigen gene of tsA58 thus obtained has a promoter of the large T-antigen gene therein, a rat into which the DNA is introduced expresses this gene (the large T-antigen gene of tsA58) in all somatic cells.

Next, the resulting DNA is introduced into totipotent cells of rats in accordance with a conventional method to prepare transgenic rats having a temperature sensitive large T-antigen gene in all cells. As a totipotent cell, ES cells having totipotency can be given in addition to fertilized ova and early embryos. A microinjection method, electropolation method, liposome method, calcium phosphate method, and the like can be used for introducing DNA into ova and cultured cells.

Furthermore, the present gene can be introduced into ova by transplanting a nucleus of cultured cells into which a desired gene of the present invention has been introduced in enucleation unfertilized ova and initializing the ova (nuclear .transplantation). However, as far as the efficiency of obtaining a transgenic rat is concerned, a transgenic rat having a large T-antigen gene of tsA58 incorporated into chromosomes of cells of each tissue at the time of development of individuals can be efficiently obtained by producing ova through microinjection of the gene of the present invention into male pronucleus of the pronucleus ova, transplanting the ova into the oviduct of an foster mother to obtain offspring, and selecting the of f spring having the injected gene, thereby stably obtaining individuals into which the gene of the present invention has been incorporated.

Immortalized cells can be prepared with extracting cells (primary cells) fromorgans of gene-introduced rats thus obtained and repeating subculture of the cells. The resulting cells obtain the characters that the cells have the capability of permanently proliferating at 33-37° C. and show the proper characteristics with terminating the proliferation at 39° C.

The retina is prepared from the eyeballs of this rat and cut into small pieces. The tissues are homogenized by using a taper-type homogenizer made of Teflon and the resulting slurry was centrifuged to obtain pellets. The resulting pellets are suspended in an enzyme (protease) solution and treated with the enzyme while shaking, thereby separating capillary vessels from unnecessary tissues. Pellets are obtained by centrifugation. The pellets thus obtained are suspended in a Hanks' balanced salt solution (HBSS) containing 25% bovine serum albumin to remove unnecessary tissues. Capillary vessel pellets are recovered by centrifugation. After enzyme treatment of the pellets by suspending again in the enzyme solution, the capillary vessels cut into fine pieces are inoculated in a culture dish. After subculturing two generations, colonies are formed. Colonies exhibiting a comparatively fast growth rate are isolated from surrounding cells using a penicillin cup. This procedure are repeated twice to isolate the cells of the present invention. Expression of a large T-antigen of tsA58, GLUT-1 transporter and p-glycoprotein are confirmed by the Western Blotting method, whereby the cells are identified to be the immortalized retinal capillary endothelial cells. The cells thus obtained exhibit excellent growth after 50 generation subculture at 33° C. and possess functions of retinal capillary endothelial cells.

Moreover, the brain of this rat is taken out to collect choroid plexus. The choroid plexus cut into pieces is treated with trypsin/EDTA to disperse cells. After terminating the enzymatic reaction by the addition of a culture medium containing fetal serum, the cells are collected by centrifugation and dispersed in a culture medium. The procedures of centrifugation and dispersion are repeated to wash the cells. The cells thus obtained are dispersed in a culture medium, inoculated on a culture plate, and incubated at 33° C. After subculturing three generations, colonies are formed. Colonies exhibiting a paving stone-like form inherent to epithelial cell and a comparatively fast growth rate are isolated from the surrounding cells using a penicillin cup. This procedure is repeated twice to isolate the cells originating from a single cell. The cells obtained are subjected to immunostaining to confirm localization of Na⁺—K⁺ ATPase and GLUT-1 transporter on the cell membrane using a confocal laser scanning microscopy, whereby the cells are identified. The resulting cells exhibit a large T-antigen, maintain excellent proliferating activity after 50 generation subculture at 33° C., and express Na⁺—K⁺ ATPase and GLUT-1 transporter. In particular, when the cells are cultured in a monolayer, Na⁺—K⁺ ATPase which is present on the basolateral membrane side (a serous membrane) in other epithelial cells, is locally present in the apical side of the cell membrane.

In the same manner, the brain of this rat is taken out to collect cerebrum. The cerebrum cut into small pieces is homogenized using a taper-type homogenizer made of Teflon and the resulting slurry is centrifuged using 16% dextran to obtain pellets (brain capillary fractions). The resulting brain capillary fractions are suspended in an enzyme (collagenase/dispase) solution and treated with the enzyme while shaking, thereby separating capillary vessels from unnecessary tissues. Pellets are obtained by centrifugation. The pellets thus obtained are suspended in a Hanks' balanced salt solution (HBSS) containing 16% dextran to remove unnecessary tissues. Capillary vessel pellets are recovered by centrifugation. After enzyme treatment of the pellets again by suspending in the enzyme solution, the capillary vessels cut into pieces are inoculated in a culture dish. After culture at 33° C. in a CO₂ incubator (5% CO₂-95% air, saturated humidity), confluent cells are treated with trypsin to collect and disperse. Then, the cells are subcultured. After subculturing three generations, colonies are formed. Colonies exhibiting a comparatively fast growth rate are isolated from surrounding cells using a penicillin cup. This procedure is repeated twice to isolate the cells of the present invention. Expression of a large T-antigen of tsA58, GLUT-1 transporter, and p-glycoprotein in the isolated cells are confirmed by the Western Blotting method. In addition, uptake of Dil-fluorescence-labeled cells (AcLDL) is observed by a confocal laser scanning microscopy to confirm expression of a scavenger receptor, and the alkaline phosphatase activity and γ-glutamyltrans peptidase activity are measured, whereby the cells are identified to be the brain capillary endothelial cells. The cells thus obtained exhibit excellent growth after 50 generation subculture at 33° C. and possess functions of brain capillary endothelial cells.

### EXAMPLES

The present invention will now be described in more detail byway of examples,which are given for the purpose of explanation and should not be construed as limiting the present invention.

### Example 1

### Preparation of Transgenic Rat

A transgenic rat carrying DNA of an SV40 temperature sensitive mutant tsA58 was prepared according to the following method.

(1) Preparation of a Gene to be Introduced

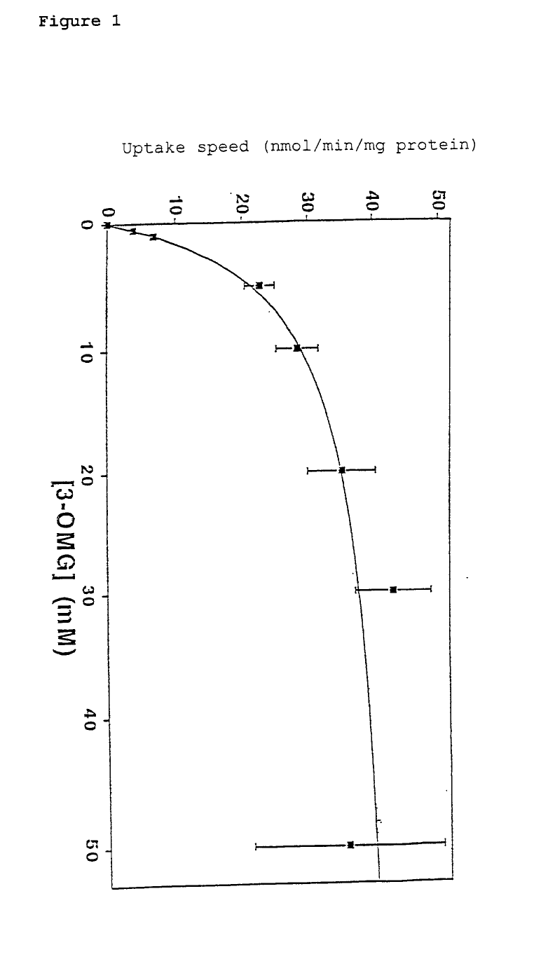

DNA of SV40 temperature sensitive mutant tsA58 was used for microinjection. The genome DNA of tsA58was linearized using a restriction endonuclease BamHI and introduced into the BamH site of pBR322 to convert the Sfi I sequence to the SacII sequence, thereby obtaining a DNA clone pSVtsA58 ori(−)−2 with deletion of the SV40 ori site (replication origin) (See Ohno T. et al., Cytotechnology 7, 165-172 (1991), FIG. 1). The DNA was prepared from the pSVtsA58 ori (−)−2 according to a conventional method. Specifically, the pSVtsA58 ori(−)−2 of plasmid DNA obtained by amplification in Escherichia coli. was digested using a restriction endonucleases BamHI (made by Takara Shuzo Co., Ltd.) and the vector region was separated by agarose gel electrophoresis (1% gel; Boeringer company). Linear DNA fragment of tsA58 DNA with a length of 5240 bp were cut out from the gel. The gel was dissolved by agarase treatment (0.6 unit/100 mg gel: Agarase; Boeringer Co.). DNA was recovered by phenol-chloroform treatment and ethanol precipitation treatment. The purified DNA was dissolved in a TE buffer (10 mM Tris-HCl containing 1 mM EDTA, pH 7.6) to obtain a purified DNA solution with a concentration of 170 μg/mL. The DNA solution was diluted with a buffer (10 mM Tris-HCl containing 0.1 mM EDTA, pH 7.6) to a concentration of 5 μg/mL to obtain a DNA solution for microinjection. The resulting DNA solution was stored at −20° C. until used for microinjection.

(2) Preparation of Transgenic Rat

Microinjection of the DNA solution prepared in (1) above to the rat ova at pronucleus stage was carried out according to the following procedures. Sexually mature Wistar rats, aged eight weeks, were kept in a condition of a 12 hour light-and-shade cycle (light hours: 4:00-16:00) at 23±2° C. and RH 55±5%. The estrous cycle of female rats was observed by vaginal smear to select the hormonal treating day. A pregnant-mare serum gonadotropic hormone (pregnant mare serum gonadotropin; PMSG, manufactured by Nippon Zenyaka Co.) was intraperitoneally administered at a dose of 150 IU/kg to female rats. After 48 hours, 75 IU/kg of human chorionic gonadotropin (hCGmanufactured by Sankyo Zoki Co.) was administered thereby effecting superovulation treatment. The female and male rats were mated by being together in a cage. The ova at pronucleous stage were collected at 32 hours after the hCG administration by oviduct perfusion. AmKRB solution (Toyoda Y. and Chang M. C., J. Reprod. Fertil., 36, 9-22 (1974)) was used for the oviduct perfusion and incubation of ova. The collected (fertilized) ova were treated by an enzyme in an mKRB solution containing 0.1% hyaluronidase (Hyaluronidase TypeI-S, made by Sigma Co.) at 37° C. for 5 minutes to remove cumulus cells. After washing three times with the mKRB solution to remove the enzyme, the fertilized ova were stored in a CO₂ incubator (5% CO₂-95% air, 37° C., saturated humidity) until DNA microinjection. A DNA solution was microinjected into the male pronucleus of the rat (fertilized) ova thus prepared. 228 ova after microinjection were transplanted in nine recipients (foster mothers) and 80 pups were obtained. The integration of the DNA was analyzed with DNA prepared from tails of the rats immediately after weaning by the PCR method (primers used: tsA58-1A, 5′-TCCTAATGTGCAGTCAGGTG-3′ (corresponds to 1365-1384 sites) tsA58-1B, 5′-ATGACGAGCTTTGGCACTTG-3′ (corresponds to 1571-1590 sites)). As a result, 20 rats (6 male, 8 female, and 6 unknown sexuality) were identified to have the gene introduced. Among these rats, 11 transgenic rat lines (male lines: #07-2, #07-5, #09-6, #12-3, #19-5, female lines: #09-7, #11-6, #12-#18-5, #19-8) which survived as long as 12 weeks after elapse of the sexual maturation period were obtained. These G0 generation transgenic rats were mated with Wistar rats and established 2 lines of male founders (#07-2, #07-5) and 3 lines of female founders (#09-7, #11-6, #19-8), by confirming that the genes was introduced in germ line and transferred to next generation

### Example 2

### Isolation of Retinal Capillary Endothelial Cells

The method of Greenwood (Greenwood J. (1992) J. Neuroimmun., 39, 123-132) was modified and applied to the isolation of retinal capillary endothelial cells from the retina. Eyeballs were collected from one transgenic rat carring a large T-antigen gene of SV40 temperature sensitive mutant tsA58 described in Example 1. The eyeballs were thoroughly washed with an ice-cooled buffer solution (HBSS containing 10 mM Hepes, 100 U/mL benzylpenicillin potassium, 100 μg/mL streptomycin sulfate, 0. 5% bovine serum albumin) in a clean bench. The retinal tissue was removed and cut into pieces with a volume of 1-2 mm³. The tissue pieces were placed in a 1 mL taper-type Teflon homogenizer (WHEATON Co.). 1 mL of ice-cooled buffer solution was added and the mixture was homogenized by four up-and-down strokes to obtain a slurry. The slurry was centrifuged (600 g, 5minutes, 4° C.) to obtain pellets. The pellets were suspended in a 1 mL of enzyme solution (HBSS containing 0.01% collagenase/dispase (Boehringer Mannheim) 100 U/mL benzylpenicillin potassium, 100 μg/mL streptomycin sulfate, 20 U/mL deoxyribonuclease I, 0.147 μg/mL tosyl-lysine-chloromethylketone) and digested by beeping in a water bath with shaking at 37° C. for 30 minutes, thereby separating capillary vessels from unnecessary tissues. The enzyme treated slurry was centrifuged (600 g, 5minutes, 4° C.) to obtain pellets.

The pellets thus obtained were suspended in HBSS containing 25% bovine serum albumin to remove unnecessary tissues. The pellets of capillary vessel fraction were recovered by centrifugation (1,000 g, 15 minutes, 4° C.). The pellets were suspended again in a 1 mL enzyme solution and treated at 37° C. for 30 minutes to digest the capillary vessels into fine pieces. The enzyme treated slurry was centrifuged (600 g, 5 minutes, 4° C.) to obtain pellets. Next, the pellets obtained were dispersed in a 2 mL culture solution (DMEM containing 15 μg/mL endothelial cell growth factor, 100 U/mL benzylpenicillin potassium, 100 μg/mL streptomycin sulfate, 2.50 μg/mL amphotericin B) and inoculated in a 35 mm culture dish coated with collagen type I (a product of Becton Dickinson Co.). The cells were incubated (primary culture) at 33° C. in a CO₂ incubator (5% CO₂-95% air, saturated humidity). Subculture was carried out at an interval of about one week using a trypsin solution (0.05% Trypsin, 0.53 mM EDTA; manufactured by Gibco BRL) while replacing the medium twice a week. After subculturing twice, 10²-10³ cells were inoculated in a 100 mm φ culture dish coated with collagen type I (a product of Becton Dickinson Co.). The cells were incubated at 33° C. in a CO₂ incubator to form colonies. After preparation of colonies for 7-10 days while replacing the medium twice a week, the colonies exhibiting a comparatively fast growth rate were isolated from the surrounding cells using a penicillin cup. The cells obtained were again inoculated in a 100 mm φ culture dish and incubated at 33° C. in a CO₂ incubator to form colonies. Colonies exhibiting a comparatively fast growth rate were isolated using a penicillin cup to obtain five lines of cells (TR-iBRB2, TR-iBRB4, TR-iBRB6, TR-iBRB8, TR-iBRB9).

TR-iBRB2was deposited in National Institute of Bioscience and Human-Technology, Agency of Industrial Science and Technology, the Ministry of International Trade and Industries. The deposition number is FERM BP-6507.

### Example 3

### Confirmation of Large T-antigen Proteins

Expression of large T-antigen proteins in the five cell lines obtained in Example 2 was examined by the Western Blotting method (Experimental Medicine Separate Volume, Biotechnology Manual UP Series, “Cancer research protocol by the molecular biological approach”, pages 108-115, YODOSHA Publishing Co., 1995). The five cell lines (the 20^{th} generation) were cultured in90 mm φ culture dishes until saturation. The collected cells were solubilized using 3% SDS-PBS (pH 7.4) and unsolubilized fractions were removed by centrifugation at 10,000 rpm for 10 minutes, and then the total amount of proteins was determined by the Bradford method using the protein assay kit II of the BIO-RAD Co. The proteins were separated by the SDS polyacrylamide gel electrophoresis in the amount of 20 μg each and transferred onto nitrocellulose membranes. The nitrocellulose membranes blocked by a 3% skimmed milk solution were reacted with an anti-SV40 large T-antigen mouse antibody (DP02-C, CALBIOCHEMCO.), as a primary antibody, and a HRP labeled anti-mouse IgG antibody (Amersham Co.), as a secondary antibody, to detect the reactions specific to large T-antigen proteins using the ECL Western Blotting detection system (RPN2106M1, a product of Amersham Co.). The results are shown in Table 1. In Table, “+” indicates that the reaction specific to a large T-antigen protein was detected. As a result, the expression of large T-antigen proteins was confirmed in all five cell lines.

TABLE 1

| | | | | | |
|---|---|---|---|---|---|
| Cells | TR-iBRB2 | TR-iBRB4 | TR-iBRB6 | TR-iBRB8 | TR-iBRB9 |
| T-Antigen | + | + | + | + | + |

### Example 4

### Identification of Cells

The cells obtained in Example 2 were identified to be retinal capillary endothelial cells by confirming the expression of a GLUTi transporter and p-glycoprotein by the Western Blotting method. Using nitrocellulose membranes prepared in the same manner as in Example 3, the cells obtained were reacted with an anti-GLUT-1 mouse antibody (Temecular, Calif., ChemiconCo.) or an anti-p-glycoprotein rabbit antibody (anti-mdr antibody, Oncogene Research Products Co.), as primary antibodies, and a HRP labeled anti-mouse IgG antibody (Amersham Co.) or a HRP labeled anti-rabbit IgG antibody (Cappel Co.), as secondary antibodies, to detect the reactions specific to GLUT-1 protein or p-glycoprotein using the ECL Western Blotting detection system (RPN2106M1, Amersham Co.). The results are shown in Table 2. In the Table, “+” means that the GLUT-1 protein or p-glycoprotein were detected. As a result the GLUT-1 protein and p-glycoprotein were detected in all five cell lines. Therefore, the five cell lines obtained were detected to be retinal capillary endothelial cells.

TABLE 2

| Cells | TR-iBRB2 | TR-iBRB4 | TR-iBRB6 | TR-iBRB8 | TR-iBRB9 |
|---|---|---|---|---|---|
| GLUT-1 | + | + | + | + | + |
| P- | + | + | + | + | + |
| Glycoprotein | | | | | |

### Example 5

### Confirmation of Glucose Transport Capability

The 3-OMG (3-o-methyl-D-glucose) uptake capability was determined using the cells TR-iBRB2 obtained in Example 2 to confirm that the cells exhibit concentration-dependent glucose transport capability, indicating the possession of a functional GLUT-1 transporter. Specifically, TR-iBRB cells were inoculated in a 24-well cell culture plate at a concentration of 3×10⁵ cells /well/mL and incubated for 24 hours at 33° C. in a CO₂ incubator to be the cells confluent. The 3-OMG uptake was determined according to the following procedure. After removing medium by aspiration, 0.2 mL of an uptake buffer containing 232 kBq/mL of [³H]3-OMG heated to 37° C. was added. The uptake buffer used in this Example did not contain glucose and was prepared from a solution which contains 122 mM NaCl, 3 mM KCl, 1.4 mM CaCl₂, 1.4 MM MgSO₄·7H₂O, 0.4 mM K₂HPO₄, 10 mM Hepes, and25mM NaHCO₃ by bubbling 5% CO₂-95% O₂ into the solution for 20 minutes and adjusting the pH of the resulting solution to 7.4 with NaOH (this is hereinafter designated as uptake buffer (1)). After 10 seconds, the uptake buffer (1) was removed and the residue was washed with the uptake buffer (1) at 4° C. The same procedure was repeated except for changing the period of time before removing the uptake buffer (1) to 20 seconds, 30 seconds, or one minutes. The cells were solubilized in 1 mL of PBS containing 1% Triton X-100 and the radioactivity was measured using a liquid scintillation counter to confirm the linearity of the 3-OMG uptake capability. As a result, an uptake time of 20 seconds was set.

Next, the substrate concentration dependency of the 3-OMG uptake capability was examined. After washing the cells with the uptake buffer (1) heated to 37° C., 0.2 mL of the uptake buffer (1) containing 462 kBq/well of [³H]3-OMG was added. Solutions containing 3-OMG at different concentrations were prepared by adding non-labeled 3-OMG to the uptake buffer (1) to make final concentrations of 0, 0.5, 1, 5, 10, 20, 30, and 50 mM. After 20 seconds, the uptake buffer (1) was removed and the residue was washed with the uptake buffer (1) containing 10 mM non-labeled 3-OMG at 4° C. Next, the cells were solubilized overnight in 1 mL of PBS containing 1% Triton X-100 and the radioactivity was measured using a liquid scintillation counter. The results are shown in FIG. 1. Using the plot formula for the uptake rate vs. the 3-OMG concentration (V=Vmax×[S]/(Km+[S]), wherein Vmax indicates a maximum velocity constant, Km indicates the Michaelis constant, and [s] is a substrate concentration), the Km and the Vmax for 3-OMG uptake were analyzed using the non-linear minimum square program (Yamaoka K. et al. (1981) J. Pharmacobio-Dyn., 4, 879-885). As a result, it was confirmed that the uptake of [³H]3-OMG which is the substrate of the GLUT-1 was concentration-dependent, the Michaelis constant (Km) was 5.6 mM, and the maximum velocity constant (Vmax) was 45 nmol/min/mg protein. Accordingly, the cells of the present invention were confirmed to exhibit a concentration-dependent glucose transport capability.

### Example 6

### Transport Capability of p-glycoprotein

Possession of a functional p-glycoprotein transport capability by cells TR-iBRB2 obtained in Example 2 was examined by measuring the uptake of cyclosporin A (CyA) which is the substrate of the p-glycoprotein and comparing the results with the uptake capability under the presence of verapamil which is a p-glycoprotein inhibitor. Specifically, TR-iBRB cells were inoculated in a 24-well cell culture plate at a concentration of 1×10⁵ cell/well/mL culture solution and incubated at 33° C. in a CO₂ incubator to be the cells confluent. The CyA uptake was determined according to the following procedure. After removing the medium by aspiration, the cells were washed with a previously heated (37° C.) uptake buffer containing glucose (which was prepared from a solution which contains 122 mM NaCl, 3 mM KCl, 1.4 mM CaCl₂, 1.4 MM MgSO₄·7H₂O, 0.4 mM K₂HPO₄, 10 mM Hepes, 25 mM NaHCO₃, and 10 mM D-glucose by bubbling 5% CO₂-95% O₂ into the solution for 20 minutes and adjusting the pH of the resulting solution to 7.4 with NaOH (this is hereinafter designated as uptake buffer (2)). After the addition of 0.2 mL of uptake buffer (2) containing 0.25% DMSO heated to 37° C., the cells were preincubated for 30 minutes. Then, the uptake buffer (2) was removed and 0.2 mL of uptake buffer (2) containing 37 kBq/mL of [³H]CyA, 0.075 μM of non-labeled CyA, and 0.25% DMSO, heated to 37° C., was added. For the measurement of uptake in the presence of verapamil, 0.2 mL of uptake buffer (2) containing 100 μM of verapamil and 0.25% DMSO heated to 37° C. was added, and the cells were preincubated for 30 minutes, followed by the removal of the uptake buffer (2) and the addition of 0.2 mL of uptake buffer (2) containing 37 kBq/mL [³H]CyA, 0.075 μM non-labeled CyA, 100 μM verapamil, and 0.25% DMSO, heated to 37° C. Both uptake reactions were carried out for 30 minutes. After removing the reaction solution, the residue was washed with uptake buffer (2) at 4° C. three times, and cells were solubilized over night with the addition of 1 mL of 1N NaOH. Then, the radioactivity was determined using a liquid scintillation counter. As a result, a significant increase in the uptake amount of about 1.8 times was confirmed. Specifically, the cell/medium uptake ratio of [³H]CyA which is the substrate of the p-glycoprotein was 270 μL/mg protein, whereas the cell/medium uptake ratio of [³H]CyA in the presence of 100 μM verapamil which is an inhibitor of the p-glycoprotein was 490 μL/mg protein. The same results were obtained with other cells.

### Example 7

### Confirmation of Function of Scavenger Receptor

Possession of a functional scavenger receptor in cells of TR-iBRB2 obtained in Example 2 was examined by measuring uptake of an acetylated LDL (Dil-Ac-LDL, Biomedical Technologies, Stoughton, Mass.) labeled with a fluorescence labeling material, 1,1′-dioctadecyl-3,3,3′,3′-tetramethyl-indocarbocyanine perchlorate. Specifically, TR-iBRB2 cells were inoculated on a cover glass at a concentration of 1×10⁵ cells/well/mL medium and incubated at 33° C. in a CO₂ incubator for 48 hours to be the cells confluent. The Dil-Ac-LDL uptake was determined according to the following procedure. After removing the medium by aspiration, the cells were washed with the uptake buffer (2) previously heated to 37° C. Next, 0.2 mL of the uptake buffer (2) containing 10 μg/200 μL Dil-Ac-LDL which was heated to 37° C. was added, followed by incubation in a CO₂incubator for 30 minutes. After 4 hours, the uptake buffer (2) was removed and the residue was washed with the uptake buffer (2) at 4° C. After the addition of 3% form aldehyde/PBS and immobilization by allowing to stand at room temperature for 20 minutes, fluorescence uptaken into cells were measured using a confocal laser scanning microscopy. As a result, uptake of an acetylated LDL (Dil-Ac-LDL) labeled with 1,1′-dioctadecyl-3,3,3′,3′-tetramethyl-indocarbocyanine perchlorate, which is a scavenger receptor ligand, into the cells was confirmed. The same results were obtained with other cells.

### Example 8

### Isolation of Choroid Plexus Epithelial Cells

In a clean bench, the brain was collected from one transgenic rat carring a large T-antigen gene of SV40 temperature sensitive mutant tsA58 obtained in Example 1. The choroidplexus from the inner wall of the right and left ventriculus lateralis through the upper wall of the third ventricle of the brain was collected and thoroughly washed with PBS. The tissue was cut into pieces with a volume of 1-2 mm³ in 2 mL of ice-cooled PBS. The tissue pieces were suspended into 1 mL of a 10X trypsin/EDTA solution (0.5% Trypsin, 0.53 mM EDTA; manufactured by Gibco BRL) to digest by the enzyme treatment at 37° C. for 20 minutes. The tissue pieces were dispersed by gently stirring from time to time. The resulting cells were washed with a culture medium (DEME solution containing 10% FCS, 100 U/mL benzylpenicillin potassium, and 100 μg/mL streptomycin sulfate). The cells were dispersed in 2 mL of the culture and inoculated in a 35 mm φ culture dish (Falcon, manufactured by Becton Dickinson Co.) and incubated (primary culture) at 33° C. in a CO₂ incubator (5% CO₂-95% air, saturated humidity). Subculture was carried out at an interval of about one week using a trypsin/EDTA solution (0.05% Trypsin, 0.53 mM EDTA; manufactured by Gibco BRL) while replacing the medium twice a week. After subculture three times, 10²-10³ cells were inoculated in a 10 cm φ culture dish and incubated in a CO₂ incubator at 33° C. to form colonies. After 7-10 days while replacing the medium twice a week, the colonies consisting of cells having a paving stone-like form inherent to epithelial cells which exhibit a comparatively fast growth rate were isolated from the surrounding cells using a penicillin cup. The cells which were obtained were again inoculated in a 10 cm φ culture dish and incubated at 33° C. in a CO₂ incubator to form colonies. Colonies exhibiting a comparatively fast growth rate were isolated using a penicillin cup to obtain five lines of cells (TR-CSFB1, TR-CSFB2, TR-CSFB3, TR-CSFB4, TR-CSFB5).

TR-CSFB3 was deposited in National Institute of Bioscience and Human-Technology, Agency of Industrial Science and Technology, the Ministry of International Trade and Industries. The deposition number is FERM BP-6508.

### Example 9

### Confirmation of Large T-antigen Proteins

Expression of large T-antigen proteins in the five cell lines obtained in Example 8 were examined by the Western Blotting method (Experimental Medicine Separate Volume, Biotechnology Manual UP Series, “Cancer research protocol by the molecular biological approach”, pages 108-115, YODOSHA Publishing Co., 1995). The five cell lines (the 10^{th} generation) were cultured in a 90 mm φ culture dishes until saturation. The collected cells were solubilized using 1 mL of 3% SDS-PBS (pH 7.4) and unsolubilized fractions were removed by centrifugation at 10,000 rpm for 10 minutes, and then the total amount of proteins was determined by the Bradford method using the protein assay kit II of BIO-RAD Co. The proteins were separated by the SDS polyacrylamide gel electrophoresis in the amount of 20 μg each and transferred onto nitrocellulose membranes. The nitrocellulose membranes blocked by a 3% skimmed milk solution were reacted with an anti-SV40 large T-antigen mouse antibody (DP02-C, CALBIOCHEMCO.), as a primary antibody, and a HRP labeled anti-mouse IgG antibody A (Amersham Co.), as a secondary antibody, to detect the reactions specific to large T-antigen proteins using the ECL Western Blotting detection system (RPN2106M1, a product of Amersham Co.). The results are shown in Table 3. As a result, the large T-antigen proteins were detected in all five cell lines.

TABLE 3

| | TR- | TR- | TR- | TR- | TR- |
|---|---|---|---|---|---|
| Cells | CSFB1 | CSFB | CSFB3 | CSFB4 | CSFB5 |
| T-Antigen | + | + | + | + | + |

### Example 10

### Confirmation of Transport Carrier of Na⁺—K⁺ ATPase and GLUT-1

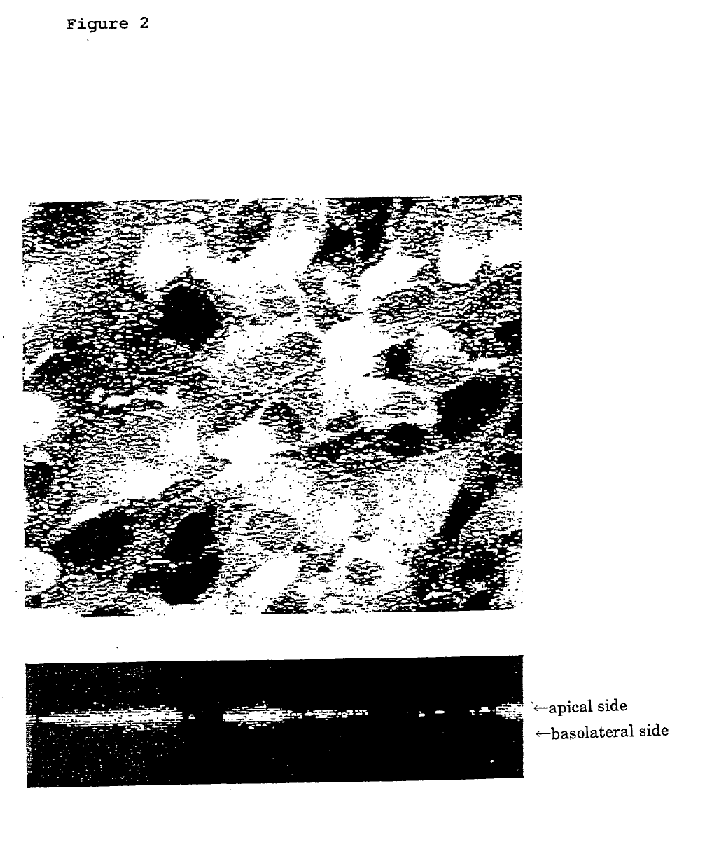

The cells obtained were cultured in a mono-layer and expression of Na⁺—K⁺ ATPase and GLUT-1 transporter on the cell membrane was confirmed by confocal laser scanning microscopy observation of immunologically stained cells. the TR-CSFB3 cells obtained in Example 8 were cultured on a collagen coated cover glass in a 35 mm φ dish (a product of Falcon). After removal of the culture solution, the cells were thoroughly washed with PBS, then 4 mL of a fixative (PBS containing 3% paraformaldehyde and 2% sucrose) was added. After allowing to stand at room temperature for 15 minutes, the cells were thoroughly washed with PBS. 2 mL of a blocking solution (Block Ace, manufactured by Dainippon Pharmaceutical Co., Ltd.) was added and the mixture was allowed to stand for one hour at 37° C. to effect blocking, followed by the reaction with a primary antibody (anti Na⁺—K⁺ ATPase β2 rabbit antibody, a product of UBI, or anti-GLUT-1 rabbit antibody, a product of Chemicon) for one hour at room temperature. The resulting product was washed four times with PBS and reacted with a secondary antibody (FITC labeled anti-rabbit IgG, a product of Capel) for one hour at room temperature, followed by washing with PBS four times. Finally, labeled cells were sealed with a glycerol sealing solution (a 90% glycerol solution in PBS containing 0.1% (v/v) of Perma Fluor (a product of Lipshaw)) The cover glass periphery was sealed with a manicure. A confocal laser scanning microscopy (CLSM; Zwiss LSM 410, manufactured by Zwiss) was used for the observation. As a result, as shown in FIG. 2, expression of Na⁺—K⁺ ATPase and GLUT-1 transporter were detected in TR-CSFB3 cells. In particular, Na⁺—K⁺ ATPase which is present on the basolateral membrane side (a serous membrane side) in other epithelial cells was seen to be locally present in the apical side of the cell membrane, confirming that the cells are choroid plexus epithelial cells. The same results were obtained with other cells.

### Example 11

### Confirmation of Proline Transport Capability

The concentration dependency of the resulting cells on the L-proline transport was examined to determine the L-proline transport capability. This was compared with the reported values of L-proline transport capability in the choroid plexus, thereby confirming that the resulting cells have functions as the choroid plexus epithelial cells.

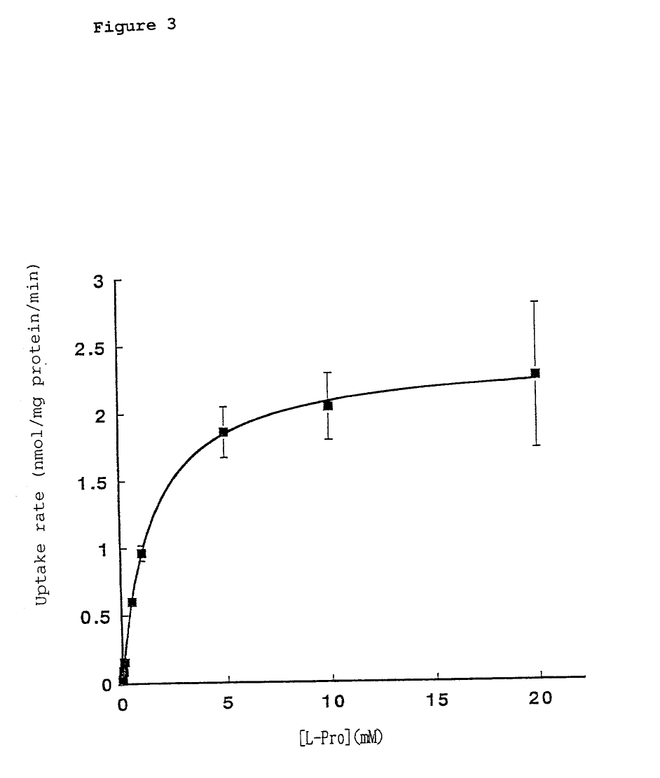

Specifically, TR-CSFB3 cells obatined in Example 8 were inoculated in a 24-well cell culture plate at a concentration of 3×10⁵ cells/well/mL and incubated for 24 hours at 33° C. in a CO₂ incubator to be the cells confluent. After removal of the medium by aspiration, the cells were washed with a previously heated (37° C.) uptake buffer (1), which was prepared from a solution which contains 122 mM NaCl, 3 mM KCl, 1.4 mM CaCl₂, 1.4 mM MgSO₄·7H₂O, 0.4 mM K₂HPO₄, 10 mM Hepes, and 25 mM NaHCO₃ by bubbling 5% CO₂-95% O₂ into the solution for 20 minutes and adjusting the pH of the resulting solution to 7.4 with NaOH. 0.2 mL of uptake buffer (1) containing 185 KBq/mL of [³H]-L-proline and heated to 37° C. was added. Solutions containing proline at different concentrations were prepared by adding non-labeled L-proline to uptake buffer (1) to make final concentrations of 0.005, 0.01, 0.05, 0.1, 0.5, 1, 5, 10, 20 mM. After the uptake reaction for 30 minutes and washing three times with PBS, 1 mL of PBS containing 1% Triton X-100 was added and the mixture was allowed to stand overnight to solubilize the cells. The radioactivity was measured using a liquid scintillation counter (LS-6500 made by Beckmann Co.). In addition, the amount of proteins was determined using a protein assay kit manufactured by Bio-Rad Co. Using the plot formula for the uptake rate vs. the L-proline concentration (V=Vmax×[S]/(Km+[S]), wherein Vmax indicates a maximum velocity constant, Km indicates the Michaelis constant, and [s] is a substrate concentration), the Km and the Vmax for L-proline uptake were analyzed using the non-linear minimum square program (Yamaoka K. et al. (1981) J. Pharmacobio-Dyn., 4, 879-885). The results are shown in FIG. 3. As a result, it was confirmed that the uptake of L-proline ([³H]-L-proline) was concentration-dependent, the Km was 1.5 mM, and the Vmax was 2.4 nmol/min/mg protein. The value for Km as determined was similar to the Km value from rabbit choroid plexus of 1.1 mM (Coben L. A. etal. (1972) Brain Res.,30, 67-82). This confirms that the resulting cells possess the function of choroid plexus epithelial cell line.

### Example 12

### Inhibition of Proline Active Transport by Choline and Ouabain

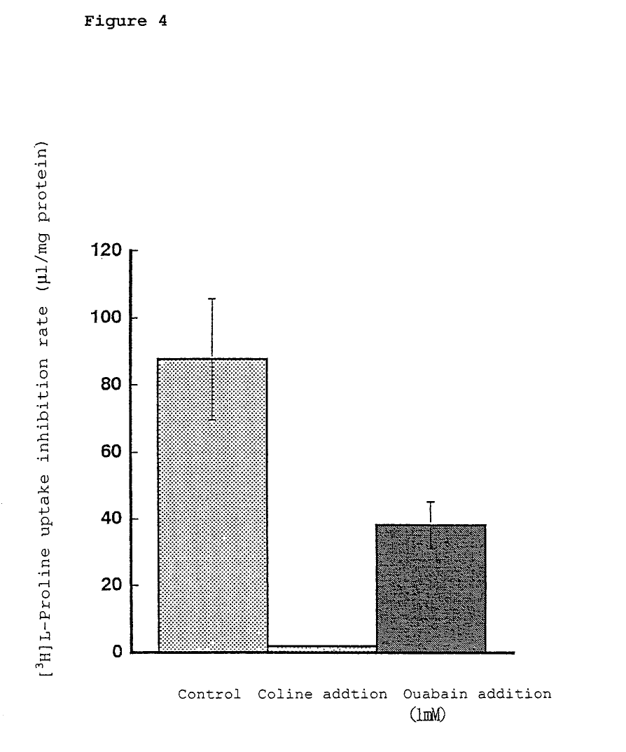

The L-proline uptake into the isolated choroid plexus is dependent on Na⁺. Therefore, the Na⁺ dependency of the L-proline uptake by the cells obtained was confirmed, and then the cells were confirmed to have functions as the choroid plexus epithelial cells in the same way as in Example 11. However, because the experiment must be carried out under Na⁺-free conditions, all Na⁺ in the uptake buffer (1) was replaced with coline. For the confirmation of the effect of ouabain, the uptake buffer (1) containing a tracer to which 1 mM of ouabain was added was used (because ouabain is an inhibitor of Na⁺—K⁺ ATPase, the concentration gradient of Na⁺ is disappeared.). Both reactions were carried out for 30 minutes. The results are shown in FIG. 4. It was confirmed that L-proline uptake was inhibited as much as 98% under Na⁺-free conditions. It was confirmed that 56% of L-proline uptake was inhibited by 1 mM ouabain. As a result, the L-proline uptake of TR-CSFB3 cells was confirmed to be Na⁺-dependent. This confirms that the resulting cells possess the function of choroid plexus epithelial cell line.

### Example 13

### Separation of Brain Capillary Endothelial Cells

Separation of capillary vessel endothelial cells from the rat brain was carried out according to the method similar to the method of Example 2. The cerebrum was collected from one transgenic rat carring a large T-antigen gene of SV40 temperature sensitive mutant tsA58 obtained in Example 1. The collected cerebrum was sufficiently washed with an ice-cooled buffer for preparation (HBSS containing 10 mM Hepes, 100 U/mL benzylpenicillin potassium, 100 μg/mL streptomycin sulfate, and 0.5% bovine serum albumin) in a clean bench, cut into pieces each having a volume of 1-2 mm³, and placed in a 1 mL taper-type Teflon homogenizer (WHEATON Co.). 1 mL of the ice-cooled buffer was added and the mixture was homogenized by four up-and-down strokes to obtain a slurry. The slurry was centrifuged (600 g, 5 minutes, 4° C.) to obtain pellets. The pellets were suspended in a 1 mL enzyme solution (HBSS containing 0.01% collagenase/dispase (Boehringer Mannheim) 100 U/mL benzylpenicillin potassium, 100 μg/mL streptomycin sulfate, 20 U/mL deoxyribonuclease I, 0.147 μg/mL tosyl-lysine-chloromethylketone)to digest with the enzyme in a water bath with shaking at 37° C. for 30 minutes, thereby separating capillary vessels from unnecessary tissues. The enzyme treated slurry was centrifuged (600 g, 5 minutes, 4° C.) to obtain pellets. The pellets thus obtained were suspended in 10 mL of HBSS containing 16% dextran to remove unnecessary tissues. The pellets of capillary vessel fractions were obtained by centrifugation (1,000 g, 15 minutes, 4° C.). The pellets were suspended again in a 1 mL of enzyme solution and treated at 37° C. for 30 minutes to digest the capillary vessels into a fine piece. The enzyme treated slurry was centrifuged (600 g, 5 minutes, 4° C.) to obtain pellets. Next, the pellets obtained were dispersed in a 2 mL of culture medium (DMEM containing 15 μg/mL endothelial cell growth factor, 100 U/mL benzylpenicillin potassium, 100 μg/mL streptomycin sulfate, 2.50 μg/mL amphotericin B) and inoculated in a 35 mm culture plate coated with collagen type I (a product of Becton Dickinson Co.). The cells were incubated (primary culture) at 33° C. in a CO₂ incubator (5% CO₂-95% air, saturated humidity). Subculture was carried out by recovering the cells using a trypsin solution (0.05% Trypsin, 0.53 MM EDTA; manufactured by Gibco BRL) and suspending the cells in a medium. The medium was replaced twice a week. Subculture was carried out at an interval of one week. After subculturing three times, 10²-10³ cells were inoculated in a 100 mm φ culture dish coated with collagen type I (a product of Becton Dickinson Co.). The cells were incubated at 33° C. in a CO₂ incubator to form colonies. After 7-10 days while replacing the medium twice a week, the colonies exhibiting a comparatively fast growth rate were isolated from the surrounding cells using a penicillin cup. The cells obtained were again inoculated in a 100 mm φ culture dish and incubated at 33° C. in a CO₂ incubator to form colonies. Colonies exhibiting a comparatively fast growth rate were isolated using a penicillin cup to obtain five lines of cells (TR-BBB1, TR-BBB5, TR-BBB6, TR-BBB11, and TR-BBB13). These cell lines exhibited a form similar to spindle fibers specific to endothelial cells.

### Example 14

### Confirmation of Large T-antigen Proteins

Expression of large T-antigen proteins in the five cell lines obtained in Example 13 was examined by the Western Blotting method (Experimental Medicine Separate Volume, Biotechnology Manual UP Series, “Cancer research protocol by the molecular biological approach”, pages 108-115, YODOSHA Publishing Co., 1995). The five cell lines (the 20^{th} generation) were cultured in a 90 mm φ culture dishes until saturation. The recovered cells were solubilized using 3% SDS-PBS (pH 7.4), unsolubilized fractions were removed by centrifugation at 10,000 rpm for 10 minutes, and then the total amount of proteins was determined by the Bradford method using the protein assay kit II of the BIO-RAD Co. The proteins were separated by the SDS polyacrylamide gel electrophoresis in the amount of 20 μg each and transferred onto nitrocellulose membranes. The nitrocellulose membranes blocked by a 3% skimmed milk solution were reacted with an anti-SV40 large T-antigen mouse antibody (DP02-C, CALBIOCHEMCo.), as a primary antibody, and a HRP labeled anti-mouse IgG antibody (Amersham Co.), as a secondary antibody, to detect the reactions specific to large T-antigen proteins using the ECL Western Blotting detection system (RPN2106M1, a product of Amersham Co.). As a result, the expression of large T-antigen proteins was detected in all five cell lines.

TABLE 4

| Cells | TR-BBB1 | TR-BBB5 | TR-BBB6 | TR-BBB11 | TR-BBB13 |
|---|---|---|---|---|---|
| T- | + | + | + | + | + |
| Antigen | | | | | |

### Example 15

### Identification of Cells

The cells obtained in Example 13 were identified to be brain capillary endothelial cells by confirming the expression of a GLUT-1 transporter and p-glycoprotein by the Western Blotting method. Using nitrocellulose membranes prepared in the same manner as in Example 14, the cells obtained were reacted with an anti-GLUT-1mouse antibody (Temecular, Calif., ChemiconCo.) or an anti-p-glycoprotein rabbit antibody (anti-mdr antibody, Oncogene Research Products Co.), as primary antibodies, and a HRP labeled anti-mouse IgG antibody (Amersham Co.) or a HRP labeled anti-rabbit IgG antibody (Cappel Co.), as secondary antibodies, to detect the reactions specific to GLUT-1 protein orp-glycoprotein using the ECL Western Blotting detection system (RPN2106M1, Amersham Co.). As a result, the GLUT-1 protein and p-glycoprotein were detected in all five cell lines. Therefore, the five cell lines obtained were identified to be brain capillary endothelial cells.

TABLE 5

| | TR- | TR- | TR- | TR- | TR- |
|---|---|---|---|---|---|
| Cells | BBB1 | BBB5 | BBB6 | BBB11 | BBB13 |
| GLUT-1 | + | + | + | + | + |
| P- | + | + | + | + | + |
| Glycaprotein | | | | | |

### Example 16

### Confirmation of Glucose Transport Capability

The 3-OMG (3-o-methyl-D-glucose) uptake capability was determined using the cells TR-BBB1, TR-BBB5, TR-BBB6, TR-BBB11, and TR-BBB13 obtained in Example 13 to confirm that the cells possess a functional GLUT-1 transport carrier, thereby confirming the concentration-dependent glucose transport capability. As a result, it was confirmed that the uptake of [³H]3-OMG which is the substrate of the GLUT-1 was concentration-dependent, and the initial uptake rate was 7.07-10.2 μl/min/mg protein.

TABLE 6

| Cells | Initial uptake rate (μ1/min/mg protein) |
|---|---|
| TR-BBB1 | 8.12 ± 0.62 |
| TR-BBB5 | 10.1 ± 1.32 |
| TR-BBB6 | 7.07 ± 0.92 |
| TR-BBB11 | 10.2 ± 0.62 |
| TR-BBB13 | 8.96 ± 0.50 |

### Example 17

### Confirmation of Function of Scavenger Receptor

Possession of a functional scavenger receptor by cells TR-BBB13 obtained in Example 13 was analyzed by measuring uptake of an acetylated LDL (Dil-Ac-LDL, Biomedical Technologies, Stoughton, Mass.) labeled with a fluorescence reagent, 1,1′-dioctadecyl-3,3,3′,3′-tetramethyl-indocarbocyanine perchlorate. The method used was done according to the method described in Example 7 was followed. Specifically, TR-BBB13 cells were inoculated on a cover glass at a concentration of 1×10⁵ cells/well/mL medium and incubated at 33° C. in a CO₂ incubator for 48 hours to be the cells confluent. For the determination of the uptake of Dil-Ac-LDL, after removing the medium by aspiration, the cells were washed with a previously heated (37° C.) uptake buffer (2), which was prepared from a solution which contains 122 mM NaCl, 3 mM KCl, 1.4 mM CaCl₂, 1.4 MM MgSO₄·7H₂O, 0.4 mMK₂HPO₄, 10 mM Hepes, 25 mM NaHCO₃, and 10 mM D-glucose by bubbling 5% CO₂-95% O₂ into the solution for 20 minutes and adjusting the pH of the resulting solution to 7.4 with NaOH. Next, 0.2 mL of the uptake buffer (2) containing 10 μg/200 μL of Dil-Ac-LDL which was heated to 37° C. was added, followed by incubation in a CO₂ incubator for 30 minutes. After 4 hours, the uptake buffer (2) was removed and the residue was washed three times with the uptake buffer (2) at 4° C. After the addition of 3% formaldehyde/PBS and immobilization by allowing to stand at room temperature for 20 minutes, fluorescence uptaken into cells were measured using a confocal laser scanning microscopy. As a result, uptake of an acetylated LDL (Dil-Ac-LDL) labeled with 1,1′-dioctadecyl-3,3,3′,3′-tetramethyl-indocarbocyanine perchlorate, which is a scavenger receptor ligand, into the cells was detected. The same results were obtained with other cells.

### Example 18

### Confirmation of Alkaline Phosphatase and γ-glutamyltranspeptidase Activities

Expression of the alkaline phosphatase and γ-glutamyltranspeptidase activities, which are expressed by the capillary endothelial cells, by the cells obtained in Example 13 were determined by a conventional method. The determination was carried out using Alkaline Phospha B-Test Wako and γ-GTP-Test Wako (manufactured by Wako Pure Chemicals Co., Ltd.) according to the standard measuring method described in the instruction manual for each kit. In addition, the amount of proteins was determined according to the Bradford method (Protein Assay Kit II manufactured by Bio-Rad Co.).

The alkaline phosphatase activity and the γ-glutamyltranspeptidase activity were found to be 8.7-25.8% and 5.4-22.6%, respectively, on the basis of the rat brain capillary vessel rich fractions as a control, thus confirming expression of enzymes specific to brain capillary endothelial cells.

TABLE 7

| | Alkaline phosphatase | |
|---|---|---|
| | activity (μU/mg | γ-Glutamyltranspeptidase |
| | protein (% of | activity (μU/mg protein (% |
| Cells | control)) | of control)) |
| TR-BBB1 | 23.7 ± 7.17 (25.8%) | 3.62 ± 0.47 (12.4%) |
| TR-BBB5 | 11.9 ± 2.92 (13.0%) | 2.05 ± 0.76 (7.0%) |
| TR-BBB6 | 22.3 ± 8.78 (24.3%) | 1.58 ± 0.52 (5.4%) |
| TR-BEE11 | 8.05 ± 2.37 (8.7%) | 6.60 ± 0.93 (22.6%) |
| TR-BBB13 | 13.7 ± 3.92 (14.9%) | 5.60 ± 1.08 (19.1%) |
| Control | 91.8 ± 30.8 (100%) | 29.2 ± 11.8 (100%) |
| (Brain | | |
| Capillaries) | | |

### INDUSTRIAL APPLICABILITY

Established cells originating from retinal capillary endothelial cells, which express a temperature sensitive SV40 large T-antigen, GLUT-1 transporter, and p-glycoprotein, are provided by the present invention. Furthermore, a method of establishing immortalized cells is provided, which comprises homogenizing the retinal tissue of a transgenic animal carring a large T-antigen gene of SV40 temperature sensitive mutant tsA58, separating capillary vessels, treating the resulting retinal capillary vessels with protease, and subculturing the resulting cells.

Such established cells of the present invention form a monolayer of the retinal capillary endothelial cells which have inside-and-outside polarity when culturing in culture dish. Therefore, the established cells are useful for predicting permeation of drugs into the retina by the assessment of drug uptake into the retinal capillary endothelial cells, studying supply and metabolism of various factors and nutritions in the retinal parenchyma, studying the transport mechanism of permeation of selective materials which are present in retinal capillary endothelial cells, studying toxicicology of drugs on retinal capillary endothelial cells, and so on. In addition, a blood retinal barrier can be reconstructed in a test tube (in vitro) by coculture with Mueller cells which are a kind of glia cells. The cell strains of the present invention are therefore useful in screening drugs regarding safety and efficacy thereof, and developing a method for diagnosing and treating diseases relating to the maintenance of intraocular homeostasis and functional disorders of retinal tissues on the cellular level studies.

Moreover, cell lines derived from choroid plexus epithelial cells are provided. The cells express a temperature sensitive SV40 large T-antigen gene, show localization of Na⁺—K⁺ ATPase and GLUT-1 transporter in the cell membrane, and when cultured in a monolayer, show the localization of Na⁺—K⁺ ATPase in the apical side. Also provided is a method of establishing immortalized cells, which derived from choroidal tissues of a transgenic animal carrying a large T-antigen gene of an SV40 temperature sensitive mutant tsA58 by protease treatment.

Due to the capability of forming tight junctions among cells when cultured in a mono-layer on a porous flat membrane and the capability of reconstructing the blood-cerebrospinal fluid barrier with a inside-and-outside polarity in vitro, the established cells are useful for studying nutrition metabolism in the brain, studying permeation of drugs into the brain, and investigating the protection mechanism of metabolism and permeation of substances into the cerebrospinal system. These cells are therefore useful in screening drugs regarding the safety and efficacy thereof, and developing a method for diagnosing and treating diseases relating to nutrition metabolism disorders and homeostatic functional disorders of the brain in cellular level studies.

Furthermore, established cells derived from brain capillary endothelial cells, which express a temperature sensitive SV40 large T-antigen, GLUT-1 transporter, and p-glycoprotein, are provided by the present invention. Still further, a method of establishing immortalized cells is provided, which comprises homogenizing the cerebrum tissue of a transgenic animal carring a large T-antigen gene of SV40 temperature sensitive mutant tsA58, separating capillary vessels, treating the resulting brain capillary vessels with protease, and subculturing the resulting cells.

Due to the capability of producing a mono-layer of the brain capillary endothelial cells when cultured on a Petri dish and of reconstructing the blood-brain barrier in vitro, the established cells are useful for studying the blood-brain barrier which restricts movement of substances to the brain tissues from blood, specifically, studying the nutrition metabolism in the brain and permeation of drugs into the brain, and investigating the protection mechanism in the blood-brain barrier. These cells are therefore useful in screening drugs regarding the safety and efficacy thereof, and developing a method for diagnosing and treating diseases relating to nutrition metabolism disorders and homeostatic functional disorders of the brain through cellular level studies.

Remarks to Deposited Microorganisms

Name and address of the organization in which the microorganisms have been deposited:

Name: National Institute of Bioscience and Human-Technology, Agency of Industrial Science and Technology, The Ministry of International Trade and Industry

Address: 1-1-3, Higashi, Tsukuba-shi, Ibaraki-ken, Japan (Postal Code: 305-3566).

Date of deposition: Sep. 18, 1998

Number of deposition given by the deposition organization: FERM BP-6507

Name and address of the organization in which the microorganisms have been deposited:

Name: National Institute of Bioscience and Human-Technology, Agency of Industrial Science and Technology, The Ministry of International Trade and Industry

Address: 1-1-3, Higashi, Tsukuba-shi, Ibaraki-ken, Japan (Postal Code: 305-3566).

Date of deposition: Sep. 18, 1998

Number of deposition given by the deposition organization: FERM BP-6508

Name and address of the organization in which the microorganisms have been deposited:

Name: National Institute of Bioscience and Human-Technology, Agency of Industrial Science and Technology, The Ministry of International Trade and Industry

Address: 1-1-3, Higashi, Tsukuba-shi, Ibaraki-ken, Japan (Postal Code: 305-3566).

Date of deposition: Sep. 22, 1999

Number of deposition given by the deposition organization: FERM BP-6873

What is claimed is:

1. An immortalized cell established from a transgenic animal into which a large T-antigen gene of SV40 temperature sensitive mutant tsA58 has been introduced.

2. The immortalized cell according to claim 1, wherein the transgenic animal is a rat.

3. An established cell derived from retinal capillary endothelial cells, which expresses a temperature sensitive SV40 large T-antigen gene, GLUT-1 transporter, and p-glycoprotein.

4. The established cell according to claim 3, having a deposition number of FERM BP-6507.

5. A method of establishing an immortalized cell which expresses a temperature sensitive SV40 large T-antigen gene, GLUT-1 transporter, and p-glycoprotein, the method comprising treating retinal capillary vessels of a transgenic animal into which a large T-antigen gene of SV40 temperature sensitive mutant tsA58 has been introduced with protease and subculturing the resulting cells.

6. An established cell which expresses a temperature sensitive SV40 large T-antigen gene, GLUT-1 transporter, and p-glycoprotein, the cell obtained by treating retinal capillary vessels of a transgenic animal into which a large T-antigen gene of SV40 temperature sensitive mutant tsA58 has been introduced with protease and subculturing the resulting cells.

7. An established cell derived from choroid plexus epithelial cells, which expresses a temperature sensitive SV40 large T-antigen gene, shows localization of Na⁺—K⁺ ATPase and GLUT-1 transporter in the cell membrane, and when cultured in a monolayer, shows the localization of Na⁺—K⁺ ATPase in the apical side.

8. The established cell according to claim 7, having a deposition number of FERM BP-6508.

9. A method of establishing an immortalized cell which expresses a temperature sensitive SV40 large T-antigen gene, shows localization of Na⁺—K⁺ ATPase and GLUT-1 transporter in the cell membrane, and when cultured in a monolayer, shows the localization of Na⁺—K⁺ ATPase in the apical side, the method comprising treating choroidal epithelium tissues of a transgenic animal into which a large T-antigen gene of SV40 temperature sensitive mutant tsA58 has been introduced with protease and subculturing the resulting cells.

10. An established cell which expresses a temperature sensitive SV40 large T-antigen gene, shows localization of Na⁺—K⁺ ATPase and GLUT-1 transporter in the cell membrane, and when cultured in a monolayer, shows the localization of Na⁺—K⁺ ATPase in the apical side, which is obtained by treating choroidal epithelium tissues of a transgenic animal into which a large T-antigen gene of SV40 temperature sensitive mutant tsA58 has been introduced with protease and subculturing the resulting cells.

11. An established cell derived from brain capillary endothelial cells, which expresses a temperature sensitive SV40 large T-antigen, GLUT-1 transporter, p-glycoprotein, alkaline phosphatase, and γ-glutamyltransferase.

12. The established cell according to claim 11, having a deposition number of FERM BP-6873.

13. A method of establishing an immortalized cell which expresses a temperature sensitive SV40 large T-antigen gene, GLUT-1 transporter, p-glycoprotein, alkaline phosphatase, and γ-glutamyltransferase, the method comprising treating brain capillary vessels of a transgenic animal into which a large T-antigen gene of SV40 temperature sensitive mutant tsA58 has been introduced with protease and subculturing the resulting cells.

14. An established cell which expresses a temperature sensitive SV40 large T-antigen gene, GLUT-1 transporter, p-glycoprotein, alkaline phosphatase, and γ-glutamyltransferase, the cell obtained by treating brain capillary vessels of a transgenic animal into which a large T-antigen gene of SV40 temperature sensitive mutant tsA58 has been introduced with protease and subculturing the resulting cells.
